(19) United States
(12) Patent Application Publication
(10) Pub. No.: US 2023/0070457 A1
(43) Pub. Date: Mar. 9, 2023

(54) MEDICAL IMAGE PROCESSING APPARATUS, X-RAY DIAGNOSTIC APPARATUS, AND STORAGE MEDIUM

(71) Applicant: CANON MEDICAL SYSTEMS CORPORATION, Otawara-shi (JP)

(72) Inventors: Yoshiteru KOBAYASHI, Sakura (JP); Yuichiro WATANABE, Yaita (JP); Kazuo IMAGAWA, Nasushiobara (JP); Mika TAKAYA, Nasushiobara (JP); Takuya AIDA, Nasushiobara (JP); Saki HASHIMOTO, Nasushiobara (JP)

(73) Assignee: CANON MEDICAL SYSTEMS CORPORATION, Otawara-shi (JP)

(21) Appl. No.: 17/929,395

(22) Filed: Sep. 2, 2022

(30) Foreign Application Priority Data
Sep. 9, 2021 (JP) 2021-147114

(51) Int. Cl. A61B 34/20; A61B 6/00; A61B 6/12; A61B 6/04; A61B 34/35; G06T 7/64; B25J 9/16; B25J 13/08; G16H 40/67

(52) U.S. Cl. CPC A61B 34/20; A61B 6/12; A61B 6/54; A61B 6/0407; A61B 6/463; A61B 6/481; A61B 6/487; A61B 6/504; A61B 6/4458; A61B 34/35; B25J 9/1664; B25J 13/08; G06T 7/64; G16H 40/67; A61B 2090/3764

(57) ABSTRACT

In one embodiment, a medical image processing apparatus includes: processing circuitry configured to extract 3D blood vessel data of an object from 3D image data of the object, detect a tip position of a medical device moving in a blood vessel in real time from a fluoroscopic image of the object inputted during an operation, and calculate at least one of a recommended route and a recommended direction of the medical device from the 3D blood vessel data, a rough route of the medical device, and the tip position of the medical device; and a terminal device configured to display a 3D blood vessel image of the object generated from the 3D blood vessel data and to designate the rough route of the medical device on the 3D blood vessel image.

### CROSS REFERENCE TO RELATED APPLICATION

This application is based upon and claims the benefit of priority from Japanese Patent Application No. 2021-147114, filed on Sep. 9, 2021, the entire contents of which are incorporated herein by reference.

### FIELD

Disclosed embodiments relate generally to a medical image processing apparatus, an X-ray diagnostic apparatus, and a storage medium of a medical image processing program.

### BACKGROUND

Interventional Radiology (IVR) is a widely performed treatment method using a medical image diagnostic apparatus such as an X-ray angiography apparatus. IVR is translated as “treatment by medical images” in Japanese. In IVR, a doctor inserts a small medical device into a blood vessel so as to diagnose and/or treat a target lesioned part of a patient, while looking through the inside of the body of the patient by using a medical image diagnostic apparatus such as an X-ray angiography apparatus, an X-ray CT apparatus, and an ultrasonic diagnostic apparatus. Medical devices inserted into blood vessels include, for example, a thin tube called a catheter, a balloon and/or a stent to be attached to the tip of the catheter, and a guidewire for guiding the catheter to a diagnosis target site and/or a treatment target site in the blood vessel.

In IVR using an X-ray angiography apparatus, while observing the time-sequential X-ray fluoroscopic images of an object (for example, a patient) generated in real time by the X-ray angiography apparatus, a doctor, i.e., a person who performs surgery, or a surgery performer, or a user, manually moves the medical device such as a guidewire and a catheter through the blood vessel of the object to reach the diagnosis target site and/or the treatment target site.

In recent years, robots for supporting catheterization procedures have also been developed. These robots, i.e., robotic support systems are under development for the purpose of performing catheterization procedures from a remote location, and/or performing fully automated or semi-automated catheterization procedures.

The original purpose of the surgery performer is to ensure that the tip of the catheter and/or guidewire reaches the diagnosis target site and/or the treatment target site. However, in the conventional manual manipulation, a check on whether the catheter interferes with the inner wall of the blood vessel, and a decision on the moving route and moving direction of the catheter in the blood vessel entirely depend on the experience and skills of the surgery performer.

Also, when performing the remote control of a catheterization procedure using the above-described robotic support system, the manipulation until the tip of the catheter and/or guidewire reaches the diagnosis target site or the treatment target site largely depends on the experience and skills of the surgery performer who manipulates the controlling device such as a joystick, a switch, and a dial on the manipulation panel provided at a remote location.

Thus, manipulation of a catheter by an inexperienced surgery performer may cause a risk such as an erroneous manipulation and delay in operation time.

In the present specification, the term “operation” is used for a surgical operation and a non-surgical operation of an object (for example, a patient), including a catheterization treatment, while the term “manipulation” is used for a manual operation by a user using a medical device, a controlling device, and an input device.

Further, in the present specification, the term “three-dimensional” may be shortly referred to as “3D”.

### BRIEF DESCRIPTION OF THE DRAWINGS

In the accompanying drawings:

FIG. 1 is a schematic diagram illustrating a configuration of a medical image processing apparatus according to the first embodiment and an X-ray diagnostic apparatus connected to the medical image processing apparatus;

FIG. 2 is a perspective view illustrating an appearance and a configuration of the X-ray diagnostic apparatus;

FIG. 3 is a schematic diagram illustrating a detailed configuration of the medical image processing apparatus according to the first embodiment;

FIG. 4 is a flowchart illustrating processing to be executed by the medical image processing apparatus according to the first embodiment;

FIG. 5A and FIG. 5B are schematic diagrams illustrating a processing concept of extracting 3D blood vessel data from 3D image data;

FIG. 6A and FIG. 6B are schematic diagrams illustrating a situation in which a rough route of a guidewire is designated on a touch panel of a terminal device;

FIG. 7A to FIG. 7C are schematic diagrams illustrating a concept of generating a display image by composing a fluoroscopic image and a 3D blood vessel image;

FIG. 8 is a schematic diagram illustrating a concept of displaying the calculated recommended route and recommended direction on a display in a manner that both are superimposed on the fluoroscopic image and the 3D blood vessel image;

FIG. 9 is schematic diagram illustrating a concept of calculating a route along a blood vessel wall at a vascular curved portion or a vascular branch portion as a recommended route;

FIG. 10 is a schematic diagram illustrating a display image that notifies a user that the current position of the guidewire is correct, when the position of the tip of the guidewire matches the recommended route;

FIG. 11 is a schematic diagram illustrating a display image that notifies the user that the current position of the guidewire is incorrect, when the position of the tip of the guidewire deviates from the recommended route;

FIG. 12A and FIG. 12B are schematic diagrams illustrating a display image indicating an alarm, when the rough route designated by the user is not a route along which the guidewire can move;

FIG. 13 is a schematic diagram illustrating a concept of controlling a table such that the tip of the guidewire is displayed at the center of the screen;

FIG. 14 is a schematic diagram illustrating a detailed configuration of a medical image processing apparatus according to a modification of the first embodiment;

FIG. 15 is a schematic diagram illustrating a display image in which a simulated image of a stent is depicted at the optimum position where the stent should be released;

FIG. 16 is a schematic diagram illustrating a configuration of a medical image processing apparatus according to the second embodiment; and

FIG. 17 is a schematic diagram illustrating a situation in which a main body of an IVR support robot is disposed near the bed.

### DETAILED DESCRIPTION

In one embodiment, a medical image processing apparatus includes: processing circuitry configured to extract three-dimensional (3D) blood vessel data of an object from three-dimensional (3D) image data of the object, detect a tip position of a medical device moving in a blood vessel in real time from a fluoroscopic image of the object inputted during an operation, and calculate at least one of a recommended route and a recommended direction of the medical device from the 3D blood vessel data, a rough route of the medical device, and the detected tip position of the medical device; and a terminal device configured to display a three-dimensional (3D) blood vessel image of the object generated from the 3D blood vessel data and designate the rough route of the medical device on the 3D blood vessel image.

Hereinafter, embodiments of the present invention will be described by referring to the accompanying drawings.

### First Embodiment

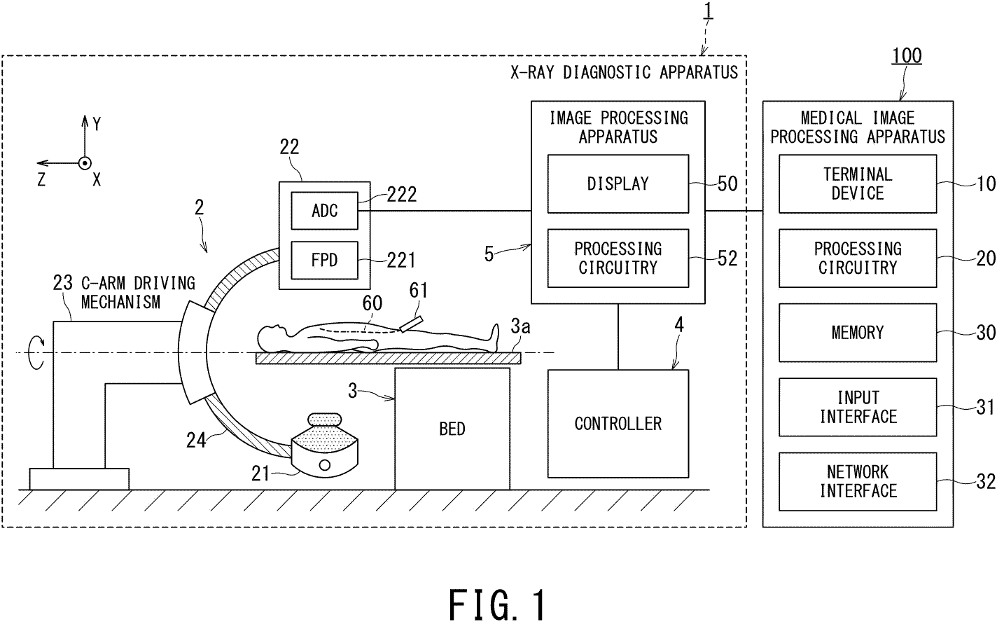

FIG. 1 is a schematic diagram illustrating a configuration of a medical image processing apparatus 100 according to the first embodiment and an X-ray diagnostic apparatus 1 connected to the medical image processing apparatus 100.

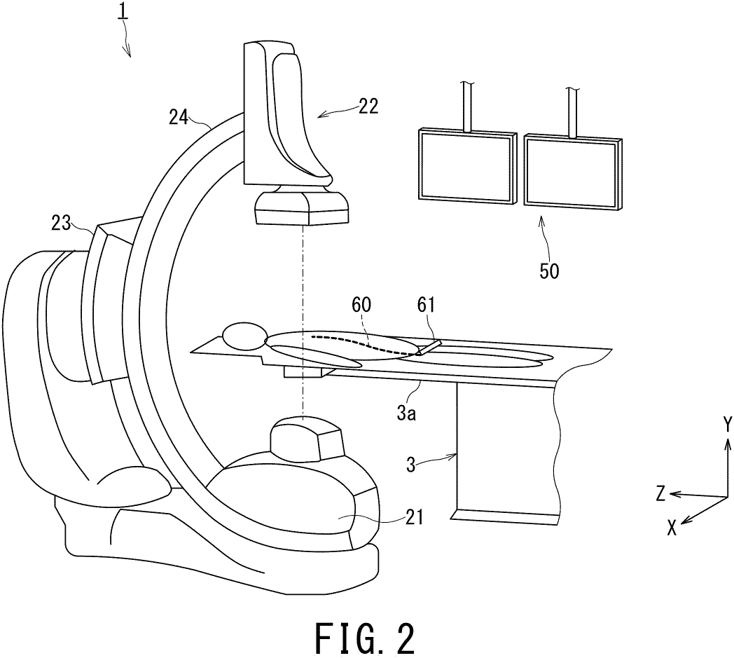

FIG. 2 is a perspective view illustrating an appearance and a configuration of the X-ray diagnostic apparatus 1.

The X-ray diagnostic apparatus 1 mainly includes a scanner 2, a bed 3, a controller 4, and a digital fluorography (DF) apparatus 5 (i.e., image processing apparatus 5). The scanner 2, the bed 3, and the controller 4 are generally installed in an operation room (i.e., examination/treatment room), and the image processing apparatus 5 is installed in a control room adjacent to the operation room.

The scanner 2 includes: an X-ray irradiator 21, an X-ray detector 22, a C-arm driving mechanism 23, and a C-arm 24.

The X-ray irradiator 21 is installed at one end of the C-arm 24. The X-ray irradiator 21 is provided so as to be able to move back and forth under the control of the controller 4. The X-ray irradiator 21 has an X-ray source (for example, an X-ray tube) and a movable aperture device. The X-ray tube receives high voltage power from a high voltage generator and generates X-rays depending on the conditions of high voltage power. The movable aperture device movably supports the aperture blades made of an X-ray shielding material at the X-ray irradiation port of the X-ray tube. A radiation quality adjusting filter for adjusting the quality of the X-rays generated by the X-ray tube may be provided on the front face of the X-ray tube.

The X-ray detector 22 is provided at the other end of the C-arm 24 so as to face the X-ray irradiator 21. The X-ray detector 22 is provided so as to be able to move back and forth under the control of the controller 4. The X-ray detector 22 includes a flat panel detector (FPD) 221 and an analog to digital converter (ADC) 222.

The FPD 221 has a plurality of detection elements arranged in two dimensions. The scanning lines and signal lines are arranged so as to be orthogonal to each other between the respective detection elements of the FPD 221. A grid may be provided on the front of the FPD 221. In order to absorb scattered rays made incident on the FPD 221 and improve contrast of an X-ray image, the grid is composed of a material having high X-ray absorption rate and another material having low X-ray absorption rate, in a manner that each of which is laminated alternately and regularly. For example, each layer made of a high X-ray absorbing material such as lead is interposed between inter-spacers made of a low X-ray absorbing material such as aluminum and wood.

The ADC 222 converts projection data of the time-sequential analog signals (i.e., video signals) outputted from the FPD 221 into digital signals, and outputs the digital signals to the image processing apparatus 5.

The X-ray detector 22 may be configured as an II (Image Intensifier)-TV system. In the II-TV system, X-rays transmitted through an object and X-rays directly made incident are converted into visible light, brightness is doubled to form sensitive projection data in the process of light-electron-light conversion, and optical projection data are converted into electrical signals by using a CCD (Charge Coupled device) image sensor.

The X-ray irradiator 21 and the X-ray detector 22 are held by the C-arm 24 so as to face each other with the object (for example, patient) interposed as the center therebetween. Under the control of the controller 4, the C-arm 24 integrally moves the X-ray irradiator 21 and the X-ray detector 22 in the arc direction of the C-arm 24 by the C-arm driving mechanism 23. Although a description will be given of the configuration in which the X-ray diagnostic apparatus 1 includes the C-arm 24 and the C-arm 24 integrally drives (i.e., works) the X-ray irradiator 21 and the X-ray detector 22, embodiments of the present invention are not limited to such a configuration. For example, the X-ray diagnostic apparatus 1 may be configured to drive the X-ray irradiator 21 and the X-ray detector 22 independently without including the C-arm 24.

Although FIG. 1 and FIG. 2 illustrate a configuration of a single-plane X-ray diagnostic apparatus 1 having only one C-arm, it may be configured as a biplane X-ray diagnostic apparatus 1 that can perform fluoroscopic imaging from two directions at the same time by using two C-arms.

The bed 3, which is supported by the floor surface, supports the table (i.e., catheter table) 3a. The bed 3 can slide the table 3a in each of the X-axis and Z-axis directions, move the table 3a up and down (i.e., in the Y-axis direction), and rotate the table 3a under the control of the controller 4. Although a description will be given of an under-tube system, in which the X-ray irradiator 21 is disposed below the table 3a in the scanner 2, the scanner 2 may be configured as an over-tube system, in which the X-ray irradiator 21 is disposed above the table 3a.

The controller 4 includes a central processing unit (CPU, not shown) and a memory (not shown). Under the control of the image processing apparatus 5, the controller 4 controls driving of the bed 3 as well as driving of the X-ray irradiator 21, the X-ray detector 22, and the C-arm 24 of the scanner 2 for alignment, i.e., positioning. Under the control of the image processing apparatus 5, the controller 4 also controls driving of respective components such as the X-ray irradiator 21, the X-ray detector 22, and the C-arm driving mechanism 23 for X-ray imaging and/or X-ray fluoroscopic imaging.

The image processing apparatus 5 is computer-based and generates an X-ray image of the object on the basis of the driving control of the entire X-ray diagnostic apparatus 1 and the signals acquired by the scanner 2. An image processing circuit 52 of the image processing apparatus 5 generates a moving image including a time-sequential X-ray fluoroscopic image from X-ray detection signals obtained in real time by imaging the object during an operation in which a medical device 60 such as a catheter is used for curing or treating the object.

The display 50 of the image processing apparatus 5 is a large display device disposed at a readily visible position to an operator such as a doctor during the operation. The display 50 displays X-ray fluoroscopic images generated by the image processing circuit 52, and also displays various support images and support information that are generated by the medical image processing apparatus 100 for supporting the operation. These support images and support information will be described below.

In FIG. 1 and FIG. 2, a medical device 60 and a catheter manipulator 61 used in the operation (i.e., catheter treatment) are also illustrated. In the present specification, a thin or fine medical device, which is inserted into a tubular tissue such as a blood vessel for diagnosing and/or treating the object, is mainly referred to as a medical device 60. As described above, a medical device inserted into a blood vessel includes, for example, a thin tube called a catheter, a balloon and/or a stent attached to the tip of the catheter, and a guidewire for leading the catheter to the diagnosis target site and/or the treatment target site in the blood vessel.

The catheter manipulator 61 is an instrument that enables the operator, such as a doctor, to manually insert the medical device 60 such as a guidewire and a catheter into a blood vessel, and to manually move the medical device 60 to a predetermined target site.

The medical image processing apparatus 100 is configured to be connectable to the X-ray diagnostic apparatus 1, and is configured as a computer such as a workstation or a personal computer, for example. The medical image processing apparatus 100 provides the operator using the medical device 60 with images and information to support the operation.

The medical image processing apparatus 100 includes, at least, a terminal device 10, processing circuitry 20, a memory 30, an input interface 31, and a network interface 32.

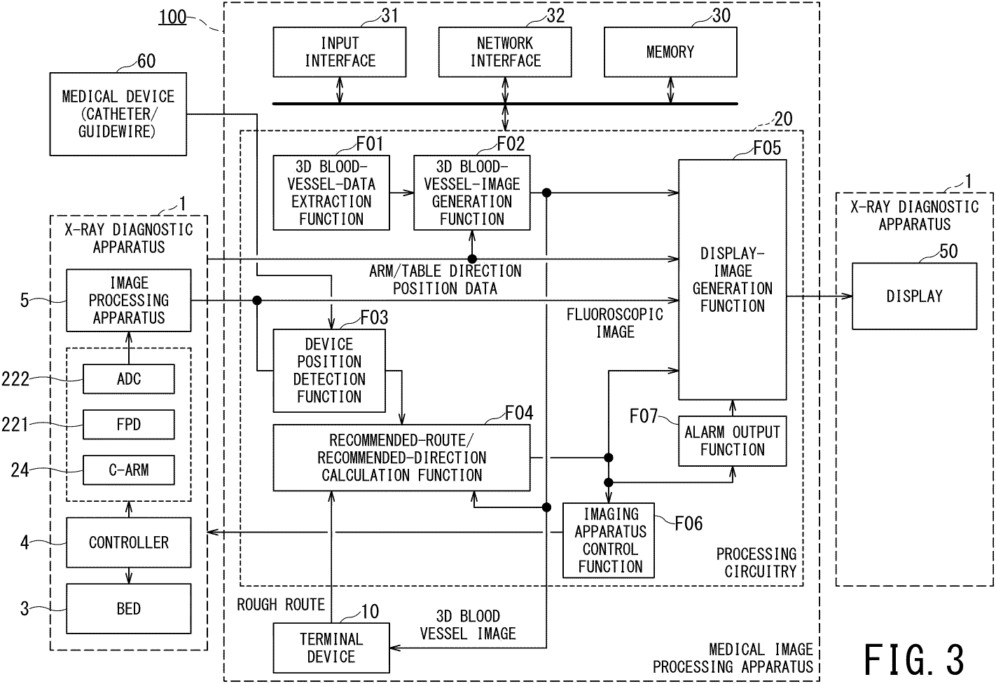

FIG. 3 is a schematic diagram illustrating a detailed configuration of the medical image processing apparatus 100 according to the first embodiment. FIG. 3 also illustrates part of the configuration of the X-ray diagnostic apparatus 1.

The terminal device 10 is a portable input/display device including a display panel and a touch panel, such as a smartphone, a tablet, or a portable personal computer, for example.

The input interface 31 includes: an input device that can be manipulated by a user; and an input circuit to which a signal from the input device is inputted. The input device may be a mouse; a keyboard; a trackball; a switch; a button; a joystick; a touch pad with which the user can perform input by touching the screen; a touch screen in which a display screen and a touch pad are integrated; a non-contact input circuit using an optical sensor; and a voice input circuit. When the input device receives an input manipulation from the user, the input circuit generates an electric signal corresponding to the input manipulation and outputs the electric signal to the processing circuitry 20.

The input interface 31 is connected to a portable memory such as a USB memory, a memory card, a magnetic disk, and an optical disk, and includes a circuit that inputs data recorded in the portable memory.

The network interface 32 is a circuit that is connected to various networks such as a hospital network and the Internet by wire or wirelessly.

The memory 30 is configured as a recording component such as a semiconductor memory element including a read-only memory (ROM) and a random access memory (RAM), a hard disk, and an optical disc. The memory 30 stores various processing programs (including an OS (Operating System) in addition to an application program) to be used in the processing circuitry 20 and data necessary for executing the programs. Further, the processing circuitry 20 can store various data such as image data inputted via the input interface 31 and/or the network interface 32.

The processing circuitry 20 includes a special-purpose or general-purpose processor and implements various functions described below by software processing in which the programs stored in the memory 30 are executed. The processing circuitry 20 may be configured of hardware such as an application specific integration circuit (ASIC) and/or a programmable logic device including a field programmable gate array (FPGA). The various functions described below can also be implemented by hardware processing using such hardware. Additionally, or alternatively, the processing circuitry 20 may implement the various functions by combining hardware processing and software processing.

The processing circuitry 20 implements each of a 3D blood-vessel-data extraction function F01, a 3D blood-vessel-image generation function F02, a device position detection function F03, a recommended-route/recommended-direction calculation function F04, a display-image generation function F05, an imaging apparatus control function F06, and an alarm output function F07.

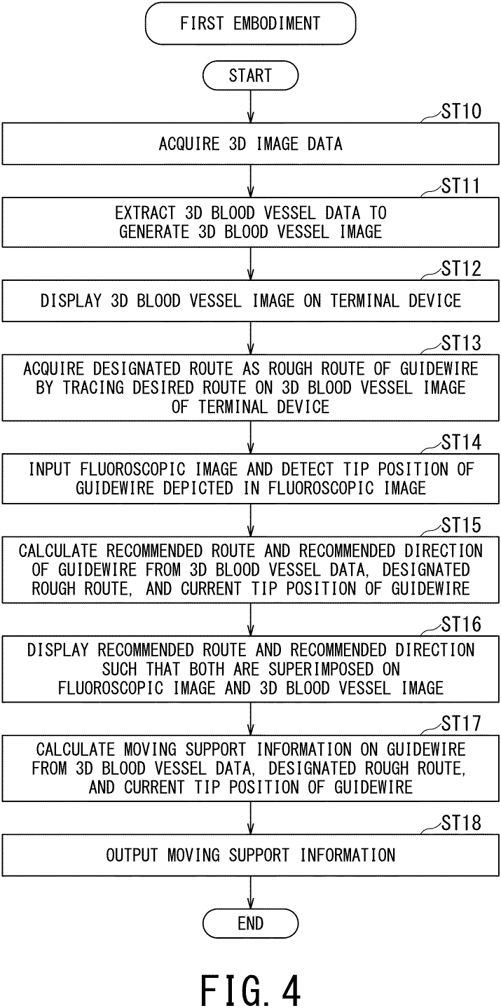

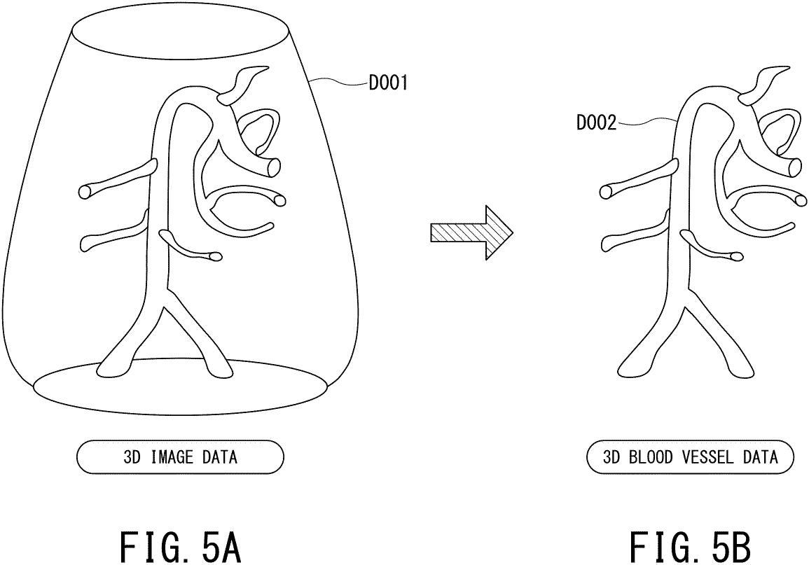

Each of these functions will be described on the basis of the flowchart of FIG. 4 and the operation conceptual diagrams of FIG. 5A to FIG. 13. FIG. 4 is a flowchart illustrating processing to be executed by the medical image processing apparatus 100 according to the first embodiment. Although the medical device 60 is described as a guidewire in the following description referring to FIG. 4 to FIG. 13, the present embodiment does not exclude the medical device 60 other than a guidewire, for example, a catheter. Thus, the term “guidewire” in the specification and/or drawings may be replaced by the term of “medical device” or “catheter”.

In step ST10 of FIG. 4, the processing circuitry 20 of the medical image processing apparatus 100 acquires 3D image data of an object to be diagnosed or treated, i.e., the object to be diagnosed or treated by using the X-ray diagnostic apparatus 1 and the medical device 60 shown in FIG. 1 and FIG. 2. The 3D image data acquired in the step ST10 are acquired in advance for the same object, and are generated by imaging the same object, for example, using a modality such as an X-ray diagnostic apparatus, an MRI apparatus, and an ultrasonic diagnostic apparatus. These 3D image data can be sent via the network interface 32 from, for example, the corresponding modality connected by the in-hospital network or from the image server in which the 3D image data are stored.

The 3D image data can also be acquired by using the X-ray diagnostic apparatus 1 before or during the diagnosis and treatment using the medical device 60. In this case, the X-ray diagnostic apparatus 1 images the object while rotating its C-arm 24 about the object, acquires the plurality of projected images, and then reconstructs the acquired projected images to obtain the 3D image data of the object.

In the next step ST11, 3D blood vessel data are extracted from the 3D image data acquired in the step ST10 by known technique. The processing of extracting the 3D blood vessel data from the 3D image data is performed by the 3D blood-vessel-data extraction function F01 in FIG. 3.

FIG. 5A and FIG. 5B are schematic diagrams illustrating a processing concept of extracting 3D blood vessel data D002 from 3D image data D001.

In the step ST11, the extracted 3D blood vessel data are further rendered from a designated direction to generate a 3D blood vessel image. The processing of generating the 3D blood vessel image from the 3D blood vessel data is performed by the 3D blood-vessel-image generation function F02 in FIG. 3.

In next step ST12, the 3D blood vessel image generated in the step ST11 is displayed on the terminal device 10. The processing of the step ST12 is also performed by the 3D blood-vessel-image generation function F02.

In the next step ST13, a rough route (i.e., approximate route or outline route) of the guidewire, which the user designates by tracing a desired route with a finger or a stylus pen on the 3D blood vessel image displayed on the terminal device 10, is acquired. The processing of acquiring the rough route designated by the user is performed by the recommended-route/recommended-direction calculation function F04 in FIG. 3.

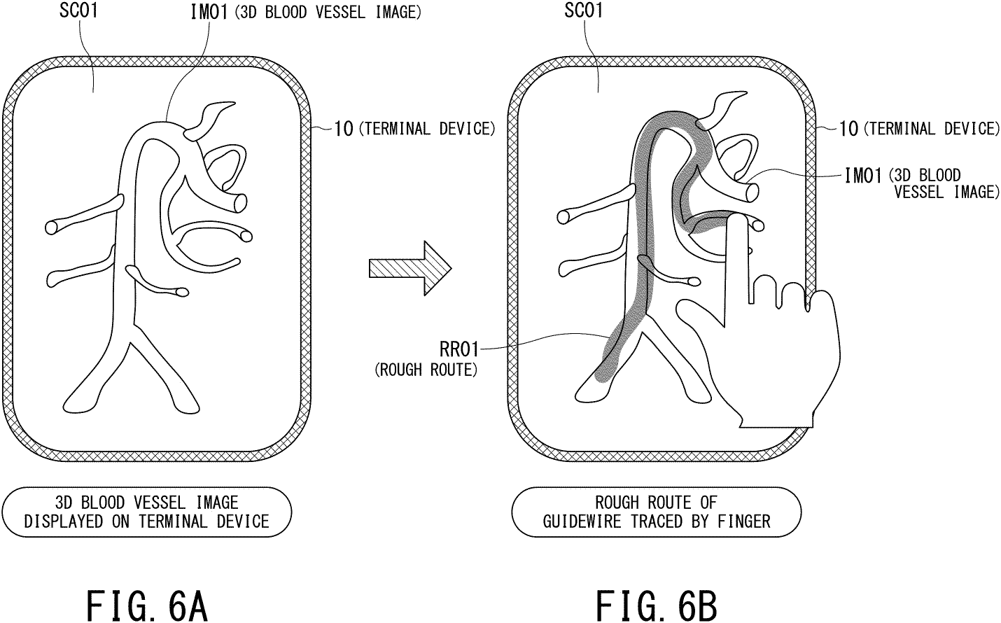

FIG. 6A and FIG. 6B are schematic diagrams illustrating a processing concept of the steps ST12 and ST13. FIG. 6A is a schematic diagram illustrating a 3D blood vessel image IM01 displayed on a display screen SC01 of the display panel/touch panel of the terminal device 10.

As illustrated in FIG. 6B, under the state where the 3D blood vessel image IM01 is displayed on the touch panel of the terminal device 10, the user can designate a rough route of the guidewire by tracing a desired route on the touch panel from the puncture site of the guidewire to the target site of the diagnosis/treatment with a finger or a stylus pen. In FIG. 6B, the rough route RR01 designated by the user is shown by a thick gray line. The route designated by the user may be literally rough and does not require fine precision.

Instead of designating the rough route, only two points including the puncture site of the guidewire and the target site of the tip of the guidewire may be designated or a plurality of points on the desired route may be designated. In such cases, the recommended-route/recommended-direction calculation function F04 may calculate the rough route from the designated two points or a plurality of points.

Returning to FIG. 4, in the step ST14, a fluoroscopic image is acquired time-sequentially, i.e., in real time from the X-ray diagnostic apparatus 1. The guidewire is depicted in each of the fluoroscopic image. The tip of the guidewire includes, for example, a member having high X-ray absorption rate such as platinum and gold. Thus, the tip of the guidewire is particularly clearly depicted in the fluoroscopic image.

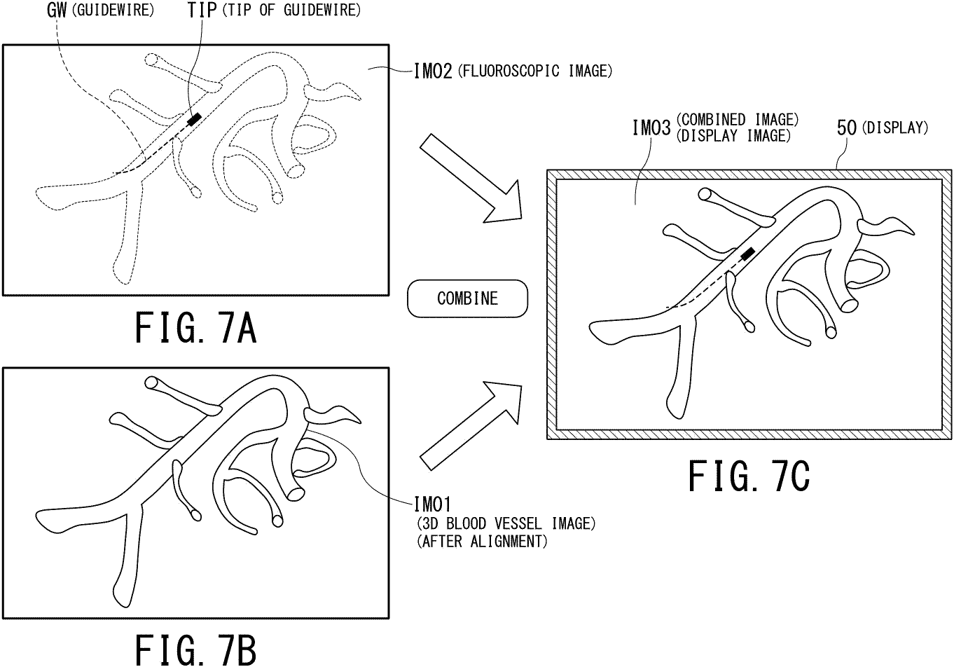

The fluoroscopic image obtained from the X-ray diagnostic apparatus 1 is inputted to the display-image generation function F05 in FIG. 3, and an image to be displayed on the display 50 is generated. Although only the fluoroscopic image can be time-sequentially displayed on the display 50, a combined image combining the fluoroscopic image acquired in real time and the 3D blood vessel image acquired in advance may be generated, so as to be time-sequentially displayed on the display 50 as shown in FIG. 7A to FIG. 7C.

FIG. 7A illustrates a fluoroscopic image IM02 obtained from the X-ray diagnostic apparatus 1. In the fluoroscopic image IM02, the guidewire GW and the tip TIP of the guidewire GW are depicted. The blood vessel may not always be clearly depicted when a contrast medium is not administered.

FIG. 7B shows the 3D blood vessel image IM01 that is aligned with the fluoroscopic image. The display-image generation function F05 acquires the data necessary for aligning (i.e., positioning) the fluoroscopic image, as exemplified by the direction and position data of the C-arm 24 and the table 3a, from the X-ray diagnostic apparatus 1 in real time, and aligns these data such that the 3D blood vessel image IM01 matches the fluoroscopic image in terms of projection direction, size, and position. Afterward, the display-image generation function F05 generates a display image IM03 by combining the aligned 3D blood vessel image IM01 and the fluoroscopic image, and causes the display 50 to display the generated display image IM03 as shown in FIG. 7C.

Further, in the step ST14 of FIG. 4, the device position detection function F03 detects the tip position of the guidewire depicted in the fluoroscopic image in real time.

In order to three-dimensionally detect the tip position of the guidewire, it is necessary to acquire fluoroscopic images that are imaged from at least two directions. For this purpose, the X-ray diagnostic apparatus 1 may rotate the C-arm 24 at predetermined intervals when the guidewire is moving forward or backward, so as to detect the tip position of the guidewire from the respective fluoroscopic images that are imaged from two directions.

In portions where the shape of the blood vessel is straight, the detection accuracy of the tip position of the guidewire does not need to be high. However, in other portions where the blood vessel curves or branches, higher detection accuracy of the tip position of the guidewire is desired. Thus, based on the 3D blood vessel data and an estimated information on the tip position of the guidewire, the recommended-route/recommended-direction calculation function F04 determines whether the tip of the guidewire is at the branch point of the blood vessel, or determines whether the tip of the guidewire is at a curve portion of the blood vessel having a curvature equal to or larger a predetermined value.

If it is determined that the tip of the guidewire is at the branch point of the blood vessel, or at a curve portion of the blood vessel having a curvature equal to or larger than a predetermined value, the imaging apparatus control function F06 causes the C-arm 24 to rotate such that fluoroscopic images can be obtained from a plurality of directions of the object.

Afterward, the device position detection function F03 three-dimensionally detects the tip position of the guidewire, based on the respective obtained fluoroscopic images that are imaged from the plurality of directions during rotation of the C-arm 24.

When the X-ray diagnostic apparatus 1 includes a scanner 2 of a biplane system, the tip position of the guidewire is three-dimensionally detected based on the fluoroscopic images that are imaged from two directions using two arms.

Further, a position sensor configured to detect 3D positional information may be provided at the tip of a medical device such as a guidewire. The position sensor may be a sensor that detects a magnetic field from a magnetic-field transmitter installed near or around the bed 3, for example. When the guidewire is provided with the position sensor at its tip, the device position detection function F03 three-dimensionally detects the tip position of the guidewire on the basis of the 3D position information outputted from the position sensor.

Returning to FIG. 4, in the step ST15, the recommended route and recommended direction of the tip of the guidewire are calculated based on the 3D blood vessel data, the designated rough route, and the current tip position of the guidewire. It is not necessary to calculate both of the recommended route and the recommended direction, and it is sufficient if either one is calculated. The processing of the step ST15 is performed by the recommended-route/recommended-direction calculation function F04 in FIG. 3.

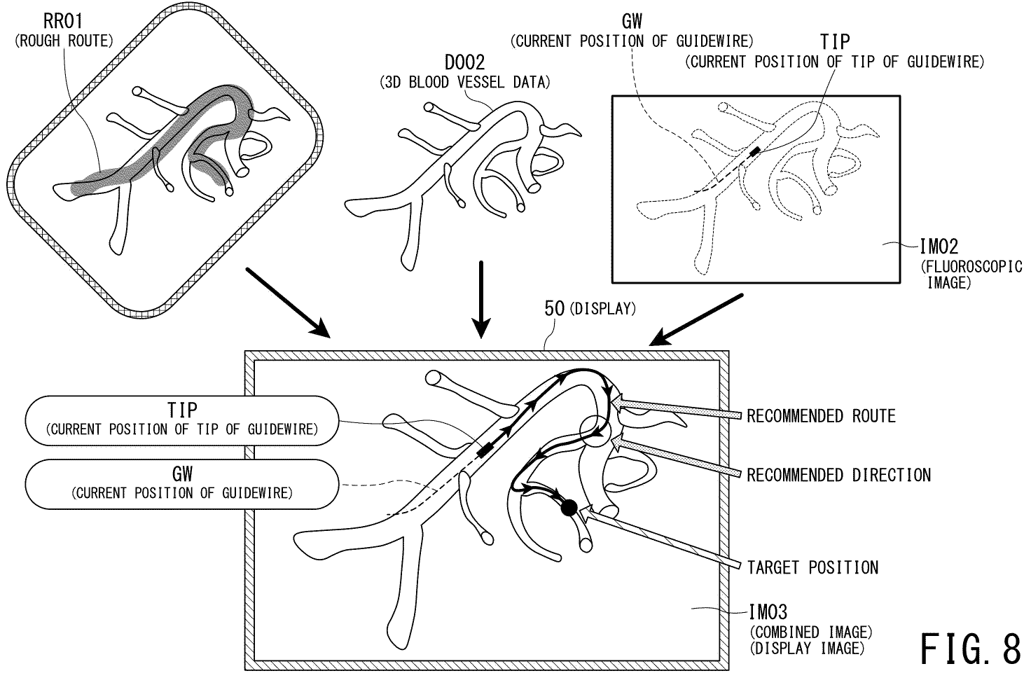

In the next step ST16, the calculated recommended route and/or recommended direction are superimposed on the fluoroscopic image and the 3D blood vessel image and shown on the display. Specifically, in the step ST16, the display image IM03 is generated by superimposing at least one of the calculated recommended route and recommended direction on the fluoroscopic image to be displayed on the display 50. Alternatively, in the step ST16, the display-image IM03 is generated by superimposing at least one of the calculated recommended route and recommended direction on the combined image combining the time-sequential fluoroscopic image and the 3D blood vessel image and shown on the display 50 as shown in the lower part of FIG. 8.

In the display-image IM03 on the display 50 shown in the lower part of FIG. 8, in addition to the image GW indicating the current position of the guidewire and the image TIP indicating the current tip position of the guidewire, the calculated recommended route of the tip of the guidewire is displayed by, for example, a thick solid line, and the recommended direction of the tip of the guidewire is displayed by, for example, a thick arrow.

The operator who manipulates the guidewire can move the guidewire while simultaneously observing, on the display 50, the current position of the tip of the guidewire and the calculated recommended route and/or recommended direction, and thus can move forward the tip of the guidewire to the target site along the recommended route reliably, quickly, and safely.

Further, when the tip position of the guidewire deviates from the recommended route, this deviation state is displayed on the display 50, so that the moving direction of the tip of the guidewire can be corrected promptly.

The recommended route and recommended direction can be calculated from the rough route designated by the user and the blood vessel shape data of the object determined from the 3D blood vessel data. For example, the centerline of each blood vessel contained in the 3D blood vessel data can be calculated from the 3D contour information of blood vessels, and the centerline closest to the designated rough route can be used as the recommended route of the guidewire. Further, the direction from the detected current position of the tip of the guidewire toward the recommended route can be set as the recommended direction of the tip of the guidewire.

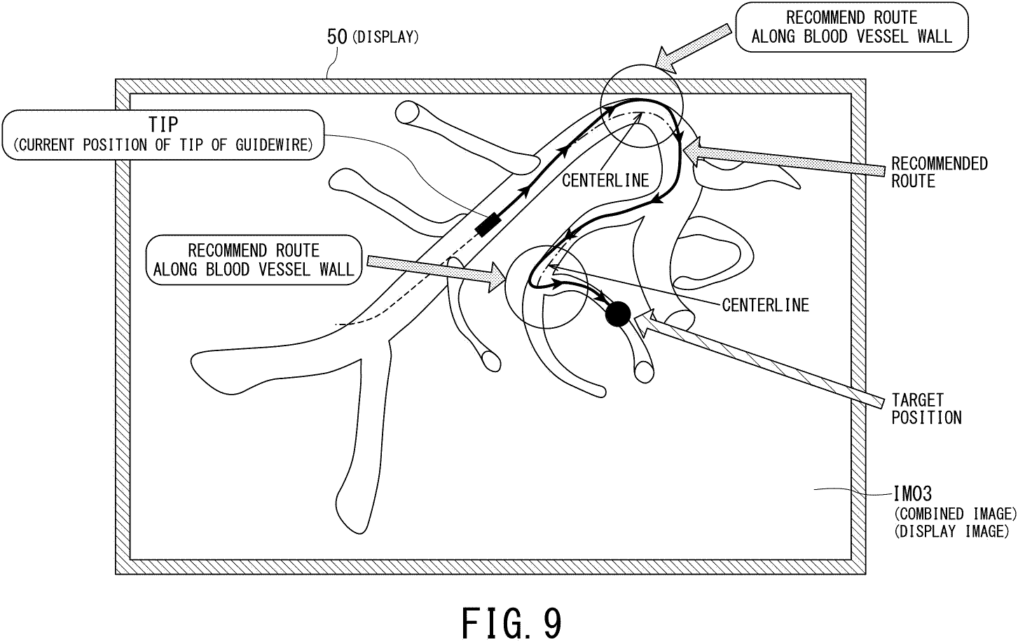

As illustrated in FIG. 9, the recommended route to be calculated by the recommended-route/recommended-direction calculation function F04 is not necessarily limited to the route along the centerline of the blood vessel. For example, as shown in the curve portion of the blood vessel at the upper part of the display image IM03 in FIG. 9, the route along the blood vessel wall may be calculated as the recommended route. For example, the recommended-route/recommended-direction calculation function F04 calculates at least one of the blood-vessel centerline and blood-vessel contour from the 3D blood vessel data to further calculate the curvature of the blood vessel, and then to calculate at least one of the recommended route and the recommended direction of the guidewire based on the calculated curvature.

In this case, for example, in the region where the calculated curvature is smaller than a predetermined value, the recommended-route/recommended-direction calculation function F04 can calculate the route along the blood-vessel centerline as the recommended route and/or calculate the direction toward the blood-vessel centerline as the recommended direction. On the other hand, in the region where the calculated curvature is equal to or larger than the predetermined value, the recommended-route/recommended-direction calculation function F04 can calculate the route or direction, in which the tip of the guidewire moves forward while contacting the wall of the blood vessel, as the recommended route or recommended direction. With such a recommended route, the guidewire can be smoothly moved without burden.

In addition, for example, in the branch portion of the blood vessel, as depicted in the central portion of the display image IM03 in FIG. 9, the route along the blood vessel wall may be calculated as the recommended route instead of the centerline of the blood vessel. For example, the recommended-route/recommended-direction calculation function F04 detects a vascular branch portion from the 3D blood vessel data and calculates the recommended route or recommended direction in the vascular branch portion in which the tip of the guidewire advances while contacting the blood vessel wall opposite to the branch blood vessel. With such a recommended route, the guidewire can be smoothly advanced to the target site without burden.

In diagnosis or treatment using the medical device 60 in some cases, due to a motion of the object, the shape of the actual blood vessel differs from the 3D shape of the blood vessel calculated from the 3D image data that are acquired in advance, such that the calculated recommended route does not match the actual case. In such case, the user can administer a contrast medium to the object by injection such that the blood vessels of each fluoroscopic image are clearly visualized.

Then, the device position detection function F03 further detects the blood vessel position (i.e., first blood vessel position) of the object during the operation from the fluoroscopic images of the blood vessel(s), into which the contrast medium is administered. If the blood vessel position (i.e., second blood vessel position) in the 3D blood vessel data differs from the first blood vessel position detected during the operation by a predetermined amount or more, the recommended-route/recommended-direction calculation function F04 updates the second blood vessel position such that the second blood vessel position matches the first blood vessel position, and calculates the recommended route and/or recommended direction of the guidewire by using the 3D blood vessel data in which the updated second blood vessel position is reflected. Such processing enables correct calculation of the recommended route and the recommended direction of the guidewire, even when the actual shape of the blood vessel is different from the shape obtained from the 3D blood vessel data due to the motion of the object.

Returning to FIG. 4, in the step ST17, in addition to the recommended route and recommended direction, the moving support information of the guidewire is calculated. The moving support information of the guidewire is also calculated from the 3D blood vessel data, the designated rough route, and the current tip position of the guidewire.

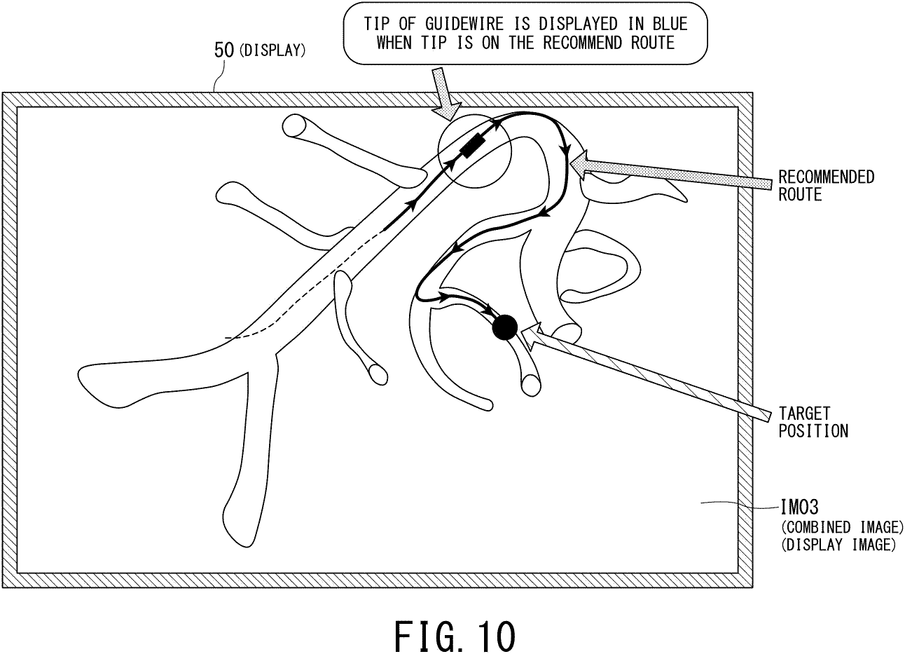

For example, when the current tip position or current moving direction of the medical device such as the guidewire matches the recommended route or recommended direction as shown in FIG. 10, the tip of the guidewire depicted in the display image on the display 50 is displayed in color such as blue or green, which notifies the user that the current position or moving direction of the guidewire is correct. Such color display is also an example of the moving support information. The processing of generating the moving support information such as the color display and superimposing the moving support information on the display image is performed by the display-image generation function F05.

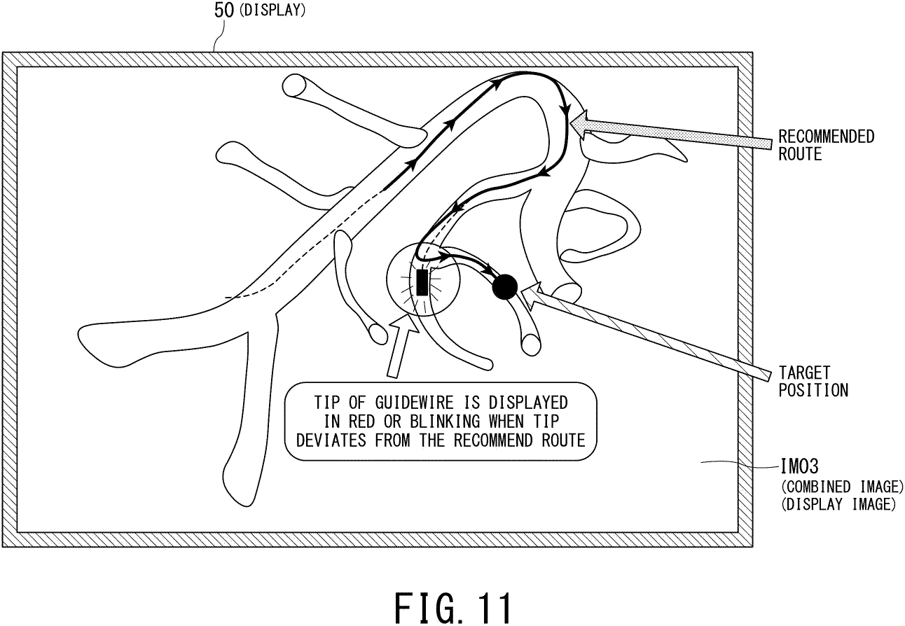

Conversely, for example, when the current tip position or current moving direction of the medical device such as the guidewire deviates from the recommended route or recommended direction as shown in FIG. 11, the alarm output function F07 (FIG. 3) outputs alarm information. The alarm information is also one example of the moving support information, where the tip of the guidewire depicted in the display image is displayed as an indicator in a conspicuous manner, for example, in red or in a blinking mode. By such an alarm display, the user can be promptly notified that the current position or current moving direction of the guidewire is incorrect. The alarm output function F07 may output the alarm information as voice by using, for example, a speaker.

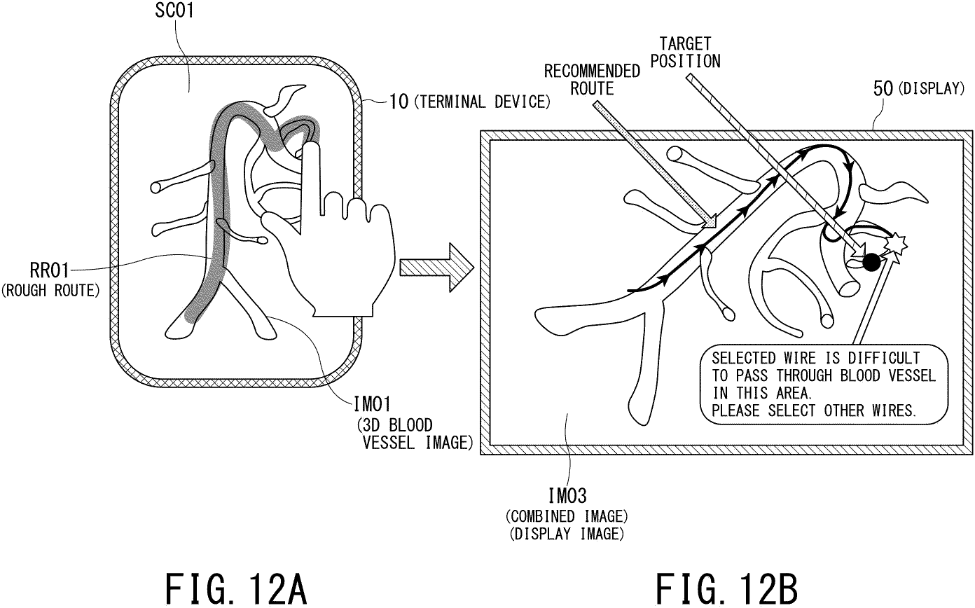

In addition, as shown in FIG. 12A and FIG. 12B, in response to designation of the rough route by the user via the terminal device 10, the alarm output function F07 may determine whether the guidewire can be moved along the designated rough route, and may output the determination result as alarm information when the determination result is negative, i.e., the designated rough route cannot be followed.

For example, as shown in FIG. 12A, the user designates the rough route RR01 via the terminal device 10. The recommended-route/recommended-direction calculation function F04 calculates the recommended route of the guidewire based on the designated rough route. At this time, the recommended-route/recommended-direction calculation function F04 further calculates the curvature of the curve portion in the blood vessel route corresponding to the designated rough route based on the blood-vessel centerline and the blood vessel contour. Then, according to information on rigidity of the guidewire acquired in advance and deformation amount of the guidewire when passing through the blood vessel curving at the calculated curvature, the recommended-route/recommended-direction calculation function F04 calculates the pressing force acting on the wall of the blood vessel when the guidewire passes through the corresponding curve portion. After that, the recommended-route/recommended-direction calculation function F04 determines whether strength of the blood vessel in the curve portion is sufficient to withstand the calculated pressing force, and applies this determination result to further determine whether the guidewire can be move along the designated rough route or not.

If the recommended-route/recommended-direction calculation function F04 determines that the guidewire cannot be moved along the designated rough route, the alarm output function F07 notifies the user of information indicating this negative determination result.

For example, as shown in FIG. 12B, to call the user’s attention, an icon such as a star-shaped icon indicating the negative determination result is displayed at the curve portion where is determined difficult to move the guidewire. Additionally or alternatively, display for prompting the user to change the currently selected guide wire to softer ones may be applied.

Further, a pressure sensor configured to detect the contact pressure between the inner wall of the blood vessel and the tip of the medical device may be attached to the tip of the medical device such as a guidewire. In this case, when the detected contact pressure exceeds a predetermined value, the alarm output function F07 may output an alarm indicating the medical device should not move forward by voice or an image on the display 50, for example.

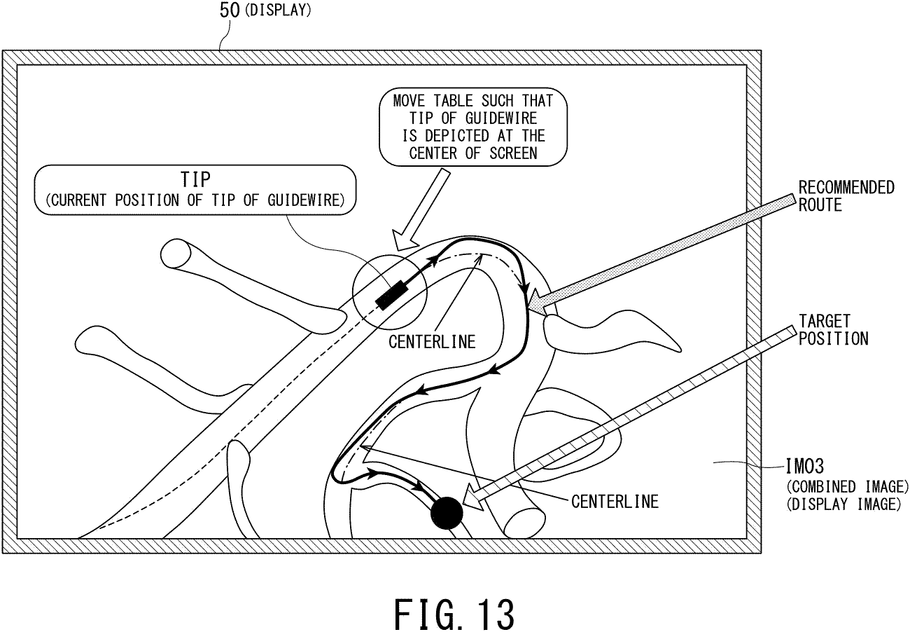

Meanwhile, as shown in FIG. 13, it is preferable that the tip of the guide wire is depicted in the center of the screen of the display 50 which enhances the operability of the operator. Thus, based on the positional information of the tip of the guidewire detected by the device position detection function F03, the imaging apparatus control function F06 shown in FIG. 3 controls the position of the bed 3 where the object is placed, such that the tip of the guidewire is always depicted at the center of the screen of the display 50 even while the guidewire is moving.

Instead of or in addition to control of the bed 3, the imaging apparatus control function F06 may control the C-arm 24 based on the positional information of the tip of the guidewire detected by the device position detection function F03 such that the tip of the guidewire is always depicted at the center of the screen of the display 50 even while the guidewire is moving.

### Modification of First Embodiment

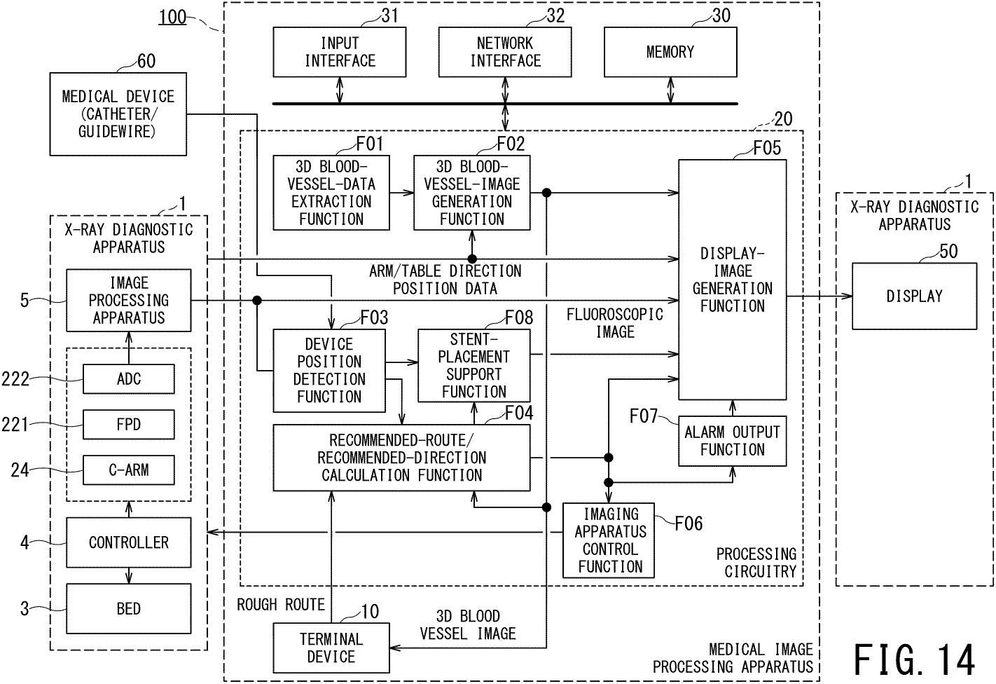

FIG. 14 is a schematic diagram illustrating a configuration of the medical image processing apparatus 100 according to a modification of the first embodiment. The modification of the first embodiment (FIG. 14) is different from the first embodiment (FIG. 3) that the processing circuitry 20 of the medical image processing apparatus 100 shown in FIG. 14 further has a stent-placement support function F08.

When the medical device used in the operation is a catheter capable of placing a stent, the user designates the placement site of the stent by pointing the corresponding position on the 3D blood vessel image displayed on the terminal device 10 with the finger, for example. In response to the designation of the placement site, the stent-placement support function F08 acquires the information of the blood vessel corresponding to the placement site from the 3D blood vessel data, and provides the user with information on recommended stents such as a stent diameter and a stent length suitable for the placement site by, for example, displaying such information via the terminal device 10 and/or the display 50.

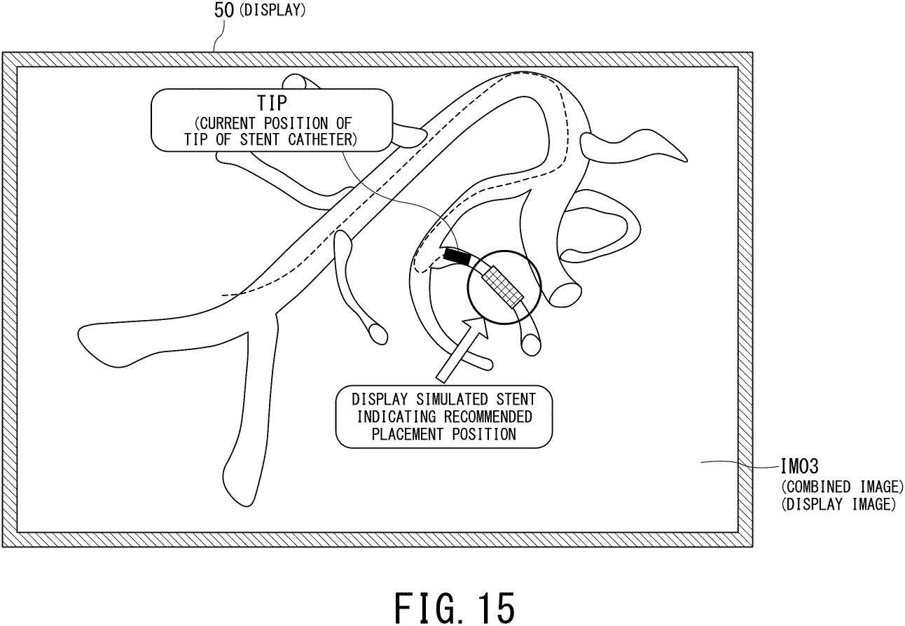

Additionally or alternatively, the stent-placement support function F08 may calculate the optimum position for releasing the stent when the tip of the catheter approaches the placement site. In this case, as shown in FIG. 15, the display-image generation function F05 may generate a simulated image for display where the stent is depicted at the position corresponding to the calculated optimum position.

### Second Embodiment

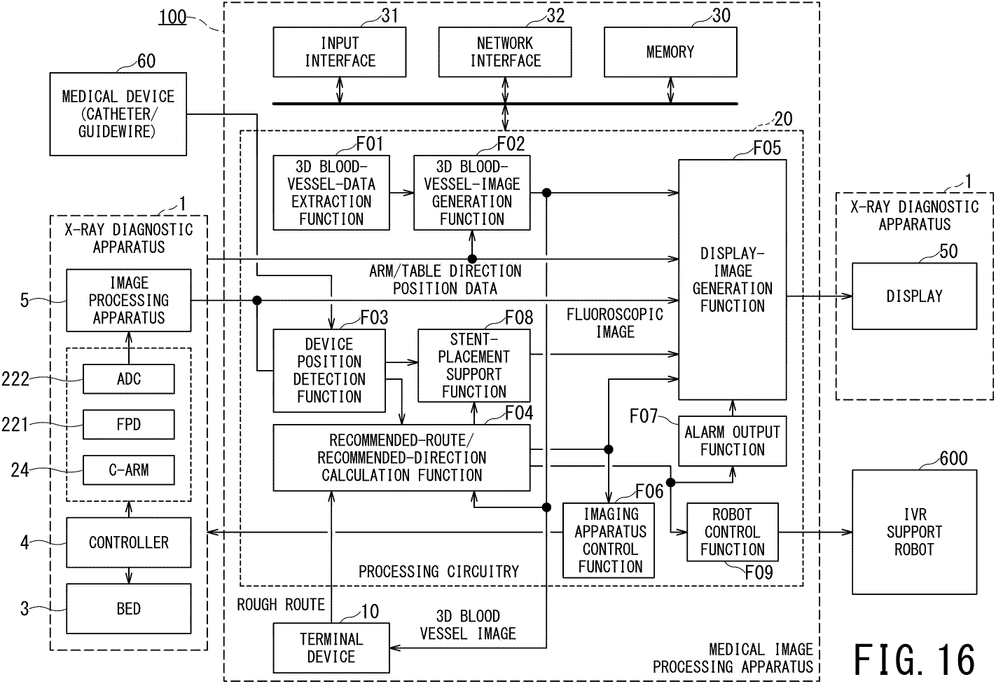

FIG. 16 is a schematic diagram illustrating a configuration of the medical image processing apparatus 100 according to the second embodiment. The second embodiment differs from the first embodiment (FIG. 3) and its modification (FIG. 14) in that the processing circuitry 20 of the medical image processing apparatus 100 in the second embodiment further has a robot control function F09. The robot control function F09 exchanges control data with the IVR support robot 600 and controls movements of the IVR support robot 600. Note that the IVR support robot 600 is provided as a separate configuration from the medical image processing apparatus 100 and the X-ray diagnostic apparatus 1.

In recent years, the IVR support robot 600 has also been made as a catheterization support robot for supporting a procedure using a catheter (IVR). The IVR support robot 600 has been developed for enabling execution of a catheterization procedure including insertion and moving forward/backward of a guidewire from a remote location, or for performing fully automated or semi-automated catheterization procedure.

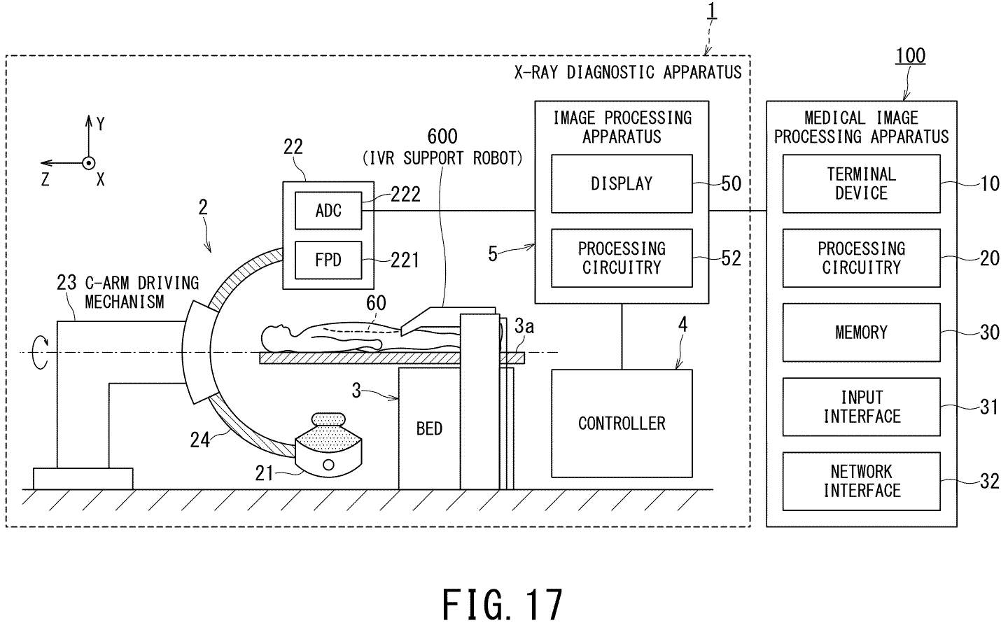

As illustrated in FIG. 17, the main unit the IVR support robot 600 as disposed near the bed 3 inserts a guidewire and/or a catheter into the object and move forward these medical devices in the blood vessel to the target site of the object. A manipulation device (for example, a manipulation panel or a control console) of the IVR support robot 600 may be disposed in a remote location different from the operation room or in the operation room.

The robot control function F09 of the second embodiment converts the calculated recommended route and recommended direction of the medical device such as a guidewire and a catheter into the control data, and outputs the control data to the IVR support robot 600. In this manner, the robot control function F09 controls the movements of the IVR support robot 600 such that the guidewire and/or the catheters are moved based on the recommended route and/or the recommended direction.

The robot control function F09 may further determine the moving speed of the medical device suitable for the object’s tissue or organ where the tip of the medical device is positioned, and control the IVR support robot 600 to move the medical device based on the determined moving speed. For example, the robot control function F09 may have the IVR support robot 600 increase the moving speed of the medical device for an organ that has very little movement like a brain, and decrease the moving speed of the medical device for an organ that has larger movement like a heart.

Additionally or alternatively, the robot control function F09 may output the control information for controlling the IVR support robot 600 to the manipulation device (for example, the manipulation panel or the control console) of the IVR support robot 600, instead of directly controlling the movements of the IVR support robot 600.

The control information for controlling the IVR support robot 600 includes, for example, torque amount and extension amount of the catheter when the IVR support robot 600 inserts the catheter into the object. By outputting such control information to the console of the IVR support robot 600, it can reduce the burden of the user who manipulates the device such as a joystick provided on the control console or manipulation panel.

According to the medical image processing apparatus of each embodiment described above, IVR-related instruments such as a guidewire and a catheter can be accurately and quickly moved to a desired position through a desired route.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
processing circuitry configured to
extract three-dimensional (3D) blood vessel data of an object from three-dimensional (3D) image data of the object,
detect a tip position of a medical device moving in a blood vessel in real time from a fluoroscopic image of the object inputted during an operation, and
calculate at least one of a recommended route and a recommended direction of the medical device from the 3D blood vessel data, a rough route of the medical device, and the tip position of the medical device; and
a terminal device configured to
display a three-dimensional (3D) blood vessel image of the object generated from the 3D blood vessel data and
designate the rough route of the medical device on the 3D blood vessel image.

2. The medical image processing apparatus according to claim 1, wherein the processing circuitry is configured to generate a display image in which at least one of the recommended route and the recommended direction is superimposed on at least one of the fluoroscopic image and a combined image and to output the display image to an external device, the combined image being an image combining the 3D blood vessel image and the fluoroscopic image.

3. The medical image processing apparatus according to claim 1, wherein the processing circuitry is configured to:
calculate at least one of a blood vessel centerline and a blood vessel contour from the 3D blood vessel data;
calculate curvature of the blood vessel from at least one of the blood vessel centerline and the blood vessel contour; and
calculate at least one of the recommended route and the recommended direction of the medical device based on the curvature of the blood vessel.

4. The medical image processing apparatus according to claim 3, wherein the processing circuitry is configured to:
calculate a route along the blood vessel centerline as the recommended route or a direction toward the blood vessel centerline as the recommended direction, in a region where the curvature is smaller than a predetermined value; and
calculate a route or direction in which a tip of the medical device moves while contacting a wall of the blood vessel as the recommended route or the recommended direction, in a region where the curvature is equal to or larger than the predetermined value.

5. The medical image processing apparatus according to claim 3, wherein the processing circuitry is configured to:
calculate pressing force by which the medical device presses against a wall of the blood vessel when the medical device passes through a curve portion of the blood vessel, using rigidity information on the medical device and deformation amount of the medical device when the medical device passes through the curve portion of the blood vessel having the calculated curvature; and
determine whether the medical device can be moved along the designated rough route or not, by determining whether strength of the blood vessel can withstand the pressing force or not.

6. The medical image processing apparatus according to claim 5, wherein, when the processing circuitry determines that the medical device cannot be moved along the designated rough route, the processing circuitry is configured to notify a user of such information.

7. The medical image processing apparatus according to claim 1, wherein:
the processing circuitry is configured to further detect a first blood vessel position of the object during the operation from a fluoroscopic image of a blood vessel in which a contrast agent is administered by a user using injector; and,
when a second blood vessel position in the 3D blood vessel data differs from the first blood vessel position detected during the operation by predetermined amount or more, the processing circuit is configured to update the second blood vessel position to match the first blood vessel position, and use the 3D blood vessel data where an updated second blood vessel position is reflected for calculating at least one of the recommended route and the recommended direction of the medical device.

8. The medical image processing apparatus according to claim 1, wherein the processing circuitry is configured to:
detect a vascular branch portion from the 3D vascular data; and
calculate a route or direction in which a tip of the medical device moves while contacting a vascular wall opposite to a branching blood vessel, as the recommended route or the recommended direction.

9. The medical image processing apparatus according to claim 2, wherein the processing circuitry is configured to:
calculate an actual position or an actual moving direction of the medical device in real time from at least one tip position of the medical device detected during the operation; and
include an indicator in the display image notifying a user that a current position or a current moving direction of the medical device is correct, when a calculated actual position of the medical device is on the recommended route or when a calculated actual moving direction of the medical device matches the recommended direction.

10. The medical image processing apparatus according to claim 9, wherein the processing circuitry is configured to depict the medical device in the display image in color to notify the user that the current position or the current moving direction of the medical device is correct.

11. The medical image processing apparatus according to claim 1, wherein:
the fluoroscopic image is an image generated by an imaging apparatus with a single-plane arm; and
the processing circuitry is configured to
rotate the single-plane arm to acquire fluoroscopic images of the object from a plurality of directions, when a tip of the medical device is determined to be at a vascular branch position by determination based on the 3D blood vessel data and information on the tip position of the medical device, or when the tip of the medical device is determined to be at a curve portion having a curvature equal to or larger than a predetermined value, and
three-dimensionally detect the tip position of the medical device based on the fluoroscopic images from the plurality of directions acquired by rotation of the single-plane arm.

12. The medical image processing apparatus according to claim 1, wherein:
a sensor configured to detect three-dimension (3D) positional information is provided at a tip portion of the medical device; and
the processing circuitry is configured to three-dimensionally detect the tip position of the medical device based on the 3D positional information outputted from the sensor.

13. The medical image processing apparatus according to claim 2, wherein the processing circuit is configured to (a) control a position and orientation of a bed on which the object is placed and/or (b) control operation of an arm supporting an X-ray irradiator and an X-ray detector for imaging the object, such that a tip of the medical device displayed on the external display is positioned at a center of the screen of the external display.

14. The medical image processing apparatus according to claim 1, wherein:
a pressure sensor configured to detect contact pressure between an inner wall of a blood vessel and a tip of the medical device is provided at the tip of the medical device; and
the processing circuitry is configured to output an alarm, when the contact pressure exceeds the predetermined value, indicating to that effect.

15. The medical image processing apparatus according to claim 1, wherein the processing circuitry is configured to:
exchange control data with an IVR support robot that controls a procedure using the medical device from a remote location, or performs the fully automated or semi-automated procedure using the medical device; and
control a movement of the IVR support robot by converting at least one of the recommended route and the recommended direction of the medical device into the control data and outputting the control data to the IVR support robot such that the medical device moves based on at least one of the recommended route and the recommended direction.

16. The medical image processing apparatus according to claim 1, wherein the processing circuitry is configured to:
exchange control data with an IVR support robot that controls a procedure using the medical device from a remote location, or performs the fully automated or semi-automated procedure using the medical device; and
determine moving speed of the medical device suitable for an object’s tissue or organ at which the tip of the medical device is positioned; and
control a movement of the IVR support robot such a that the medical device moves based on determined moving speed.

17. The medical image processing apparatus according to claim 1, wherein the processing circuitry is configured to output control information for controlling an IVR support robot to a manipulation device of an IVR support robot that controls the procedure using the medical device from a remote location, or performs the fully automated or semi-automated procedure using the medical device.

18. The medical image processing apparatus according to claim 17, wherein the control information includes at least one of torque amount of the catheter and extension amount of the catheter.

19. An X-ray diagnostic apparatus comprising the medical image processing apparatus according to claim 1.

20. A non-transitory computer-readable storage medium storing a program enabling a computer to execute processing comprising:
extracting three-dimensional (3D) blood vessel data of an object from three-dimensional (3D) image data of the object;
causing a terminal device to display a three-dimensional (3D) blood vessel image of the object generated from the 3D blood vessel data;
acquiring a rough route of the medical device moving in a blood vessel, the rough route being designated on the 3D blood vessel image displayed on the terminal device;
detecting a tip position of the medical device in real time from a fluoroscopic image of the object inputted during an operation; and
calculating at least one of a recommended route and a recommended direction of the medical device from the 3D blood vessel data, the rough route of the medical device, and the tip position of the medical device.
